(19) United States
(12) Patent Application Publication
(10) Pub. No.: US 2023/0389552 A1
(43) Pub. Date: Dec. 7, 2023

(54) A Resonance Antimicrobial Coating for Disinfecting Surface

(71) Applicant: Ah Eng Siaw, Johor Bahru (MY)

(72) Inventor: Ah Eng Siaw, Johor Bahru (MY)

(21) Appl. No.: 18/033,030

(22) PCT Filed: Oct. 23, 2020
(86) PCT No.: PCT/MY20/50117

§ 371 Date: Apr. 20, 2023

(51) Int. Cl. A01N 59/20; A01N 59/16; A01N 25/12; A01P 1/00

(52) U.S. Cl. CPC A01N 59/20; A01N 59/16; A01N 25/12; A01P 1/00

(57) ABSTRACT

A resonance antimicrobial coating composition for disinfecting surface and a method of preparation thereof are disclosed. Particularly, the resonance antimicrobial coating composition comprises a nano-sized metal oxide selected from the group consisting of silver oxide, copper oxide and a combination thereof, and at least one ultraviolet or fluorescent light-assisted photocatalyst. The atoms of the composition are in a state of energy excitation that vibrate at a frequency of 0.5 kHz to 500 kHz for a predetermined period upon being subjected to bombardment with a vibration force at the frequency for at least 24 hours.

### FIELD OF INVENTION

The invention relates to an antimicrobial coating for disinfecting surface. Particularly, the antimicrobial coating comprises atoms that are in a state of energy excitation for a period of time. A composition of the antimicrobial coating and method of preparation thereof are provided herein.

### BACKGROUND OF THE INVENTION

An emerging antimicrobial surface treatment technology is based on a resonance catalyst and photocatalytic oxidation that converts fine particles and toxic gases into safer compounds. Basically, a photocatalytic surface treatment includes the use of a photocatalyst which reacts with broad spectrum ultraviolet light to create hydroxyl radicals and super-oxide ions for oxidizing volatile organic compounds and eliminating microorganisms adsorbed on the catalyst surface.

Examples of photocatalyst commonly used as disinfectant for surface treatment include titanium oxide, titanium dioxide, zinc oxide, tungsten oxide and tungsten trioxide. By bombarding the photocatalyst with lights of certain wavelengths, electrons in the material's valence band are excited into the conduction band. As a result, the electrons are free to move and their energy can be utilized to split up nearby water and oxygen molecules into hydroxyl radicals and super-oxide ions.

Hydroxyl radicals are among the most powerful oxidizers and stronger than chlorine, ozone and peroxide but consequently the radicals are very short lived. The oxidizers can break the bonds of organic substances such as germs and volatile organic molecules into smaller compounds until only carbon dioxide and water vapor are left.

Furthermore, activated photocatalyst have shown to be capable of killing a wide range of Gram-negative and Gram-positive bacteria, filamentous and unicellular fungi, algae, protozoa, mammalian viruses and bacteriophage. However, resting stages, particularly bacterial endospores, fungal spores and protozoan cysts, are generally more resistant to photocatalytic killing than the vegetative forms, possibly due to the increased cell wall thickness. For instance, Acanthamoeba cysts and Trichoderma asperellum coniodiospores have been reported to be resistant to photocatalysis.

One improvement on photocatalytic disinfection technology includes the addition of other elements. Some examples of the elements are vanadium, copper, zinc, rhodium, silver and nickel. Great attention has been focused on using nano-silver and nano-copper particles in a coating for disinfecting the surface. It has been examined that silver and copper can provide natural anti-bacterial, anti-viral and anti-fungal benefits. When the nano-silver particles come into contact with a bacteria or virus, they suppress the cell's nutrient transport, attack the cell membrane and interfere with cell division to hinder the reproduction of the germs. Nano-copper particles, on the other hand, damage bacterial cell membranes or “envelopes” and can destroy the DNA or RNA of the microbe. Nano-copper particles generate oxidative stress on bacterial cells and create hydrogen peroxide that can kill the cells. They also interfere with proteins that play vital functions.

The invention provides an improved photocatalytic disinfection technique, a composition therefor and a method of preparing the composition.

### SUMMARY OF THE INVENTION

The primary object of the invention is to provide a resonance antimicrobial coating composition for disinfecting a surface effectively by eliminating a variety of organic contaminants and microorganisms within a short period of time.

Another object of the invention is to provide a resonance antimicrobial coating composition for disinfecting a surface in which the atoms in the composition hold sufficient vibration energy for a period of time and are able to transfer the vibration energy to surrounding organic contaminants and microorganisms upon application over the surface in order to facilitate the surface disinfection and improve the disinfection efficacy.

Still another object of the invention is to provide a resonance antimicrobial coating composition for use in disinfecting a surface in which microorganisms on the surface are induced to vibrate at resonance frequency until the cell membrane or cell wall of the microorganisms shatter and break apart.

Yet, another object of the invention is to provide a resonance antimicrobial coating composition for use in disinfecting a surface in which the antimicrobial property of the composition is achieved by contact killing as well as non-direct contact killing of microorganisms.

Still, another object of the invention is to provide a method of producing a resonance antimicrobial coating composition for disinfecting a surface in which the atoms in the composition hold sufficient vibration energy for a period of time and are able to transfer the vibration energy to surrounding organic contaminants and microorganisms upon application over the surface.

At least one of the preceding objects is met, in whole or in part, by the invention, in which the embodiment of the invention describes a resonance antimicrobial coating composition comprising a nano-sized metal oxide selected from the group consisting of silver oxide, copper oxide and a combination thereof; and at least one ultraviolet or fluorescent light-assisted photocatalyst; wherein atoms of the composition are in a state of energy excitation that vibrate at a frequency of 0.5 kHz to 500 kHz for a predetermined period upon being subjected to bombardment with a vibration force at the frequency for at least 24 hours.

In a preferred embodiment of the invention, the resonance antimicrobial coating composition further comprises a nano-sized metal oxide selected from the group consisting of titanium oxide, titanium dioxide, tungsten oxide, tungsten trioxide, zinc oxide, calcium oxide, magnesium oxide, silica, tin oxide, gold oxide, and any combination thereof.

In a preferred embodiment of the invention, the resonance antimicrobial coating composition comprises from 0.01% to 5.0% by weight of the nano-sized metal oxide.

In a preferred embodiment of the invention, the nano-sized metal oxide has a particle size ranging from 5 nm to 50 nm.

In a preferred embodiment of the invention, the ultraviolet or fluorescent light-assisted photocatalyst is titanium oxide, titanium dioxide, tungsten oxide, tungsten trioxide, zinc oxide or any combination thereof.

In a preferred embodiment of the invention, the resonance antimicrobial coating composition comprises 0.01% to 30.0% by weight of the ultraviolet or fluorescent light-assisted photocatalyst.

In a preferred embodiment of the invention, the resonance antimicrobial coating composition further comprises a binder, a liquid carrier, a surface additive, or any combination thereof.

The invention also describes a method of preparing a resonance antimicrobial coating composition comprising a nano-sized metal oxide selected from the group consisting of silver oxide, copper oxide and a combination thereof; and at least one ultraviolet or fluorescent light-assisted photocatalyst; wherein atoms of the composition are in a state of energy excitation that vibrate at a frequency of 0.5 kHz to 500 kHz for a predetermined period upon being subjected to bombardment with a vibration force at the frequency for at least 24 hours.

### BRIEF DESCRIPTION OF THE DRAWINGS

For the purpose of facilitating an understanding of the invention, there is illustrated in the accompanying drawings the preferred embodiments from an inspection of which when considered in connection with the following description, the invention, its construction and operation and many of its advantages will be readily understood and appreciated.

FIG. 1 is an agarose gel electrophoresis image of reverse transcription polymerase chain reaction (RT-PCR) products of the test samples described in Example 1.

FIG. 2 is an agarose gel electrophoresis image of reverse transcription polymerase chain reaction (RT-PCR) products of the test samples described in Example 2.

FIG. 3 is an agarose gel electrophoresis image of reverse transcription polymerase chain reaction (RT-PCR) products of the test samples described in Example 3.

### DETAILED DESCRIPTION OF THE INVENTION

One skilled in the art will readily appreciate that the invention is well adapted to carry out the objects and obtain the ends and advantages mentioned, as well as those inherent therein. The embodiment described herein is not intended as limitations on the scope of the invention.

The invention discloses a composition for disinfecting purposes, including but not limiting to surface disinfection. Particularly, the composition disclosed herein is a resonance antimicrobial coating composition having at least one nano-sized metal oxide and at least one ultraviolet or fluorescent light-assisted photocatalyst. Preferably, atoms of the composition are in a state of energy excitation in which they vibrate at a frequency of 0.5 kHz to 500 kHz for a predetermined period. It is preferable to apply the composition as a coating over a surface for effective removal of organic contaminants and inhibition of microbial growth on the surface. The composition disclosed herein is capable of both contact-killing and non-contact killing of microorganisms. Particularly, the resonance antimicrobial coating composition can be used in all indoors places such as homes, offices, hotels, airports, vehicles, etc. The composition disclosed herein is effective against a broad range of microorganisms including viruses and bacteria. Furthermore, it is capable of oxidizing odor from the air. The composition disclosed herein becomes effective within a short period of time after application. Preferably, the composition is effective after 1 min of application.

In the preferred embodiment of the invention, the resonance antimicrobial coating composition comprises at least one metal oxide selected from the group consisting of silver oxide, copper oxide, titanium oxide, titanium dioxide, tungsten oxide, tungsten trioxide, zinc oxide, calcium oxide, magnesium oxide, silica, tin oxide, and gold oxide. These metal oxides are capable of slowing or hindering microbial growth and/or inactivating or killing microbes. Preferably, these metal oxides are in the form of nano-sized particles. One skilled in the art shall not limit the metal oxide to one type of metal oxide; rather it can be a mixture of two or more types of metal oxide. High electrical conductivity metal is preferred as it can hold higher charge and therefore, higher ability and capacity to hold vibration energy of which thereafter is transferred to the coated device. In a more preferred embodiment, the resonance antimicrobial coating composition at least comprises nano-sized silver oxide, copper oxide or a combination thereof.

Silver oxide is particularly preferred over other metal oxides as silver efficiently inactivates microbes by direct contact. Nano-silver particles which come in contact with bacteria and fungi can adversely affect the cellular metabolism of these microorganisms by suppressing their cellular respiration, basal metabolism of electron transfer system and transport of substrate in the microbial cell membrane. As a result, the nano-silver particles are able to inhibit multiplication and growth of the contacted bacteria and fungi which may cause infection, odor, itchiness and sores.

Similarly, copper oxide is preferred as copper is able to efficiently inactivate microorganism by direct contact. Nano-copper particles are capable of killing microorganisms such as bacteria and viruses. Nano-copper particles damage microbial cell membranes or envelopes and destroy microbial DNA or RNA. The antimicrobial activity of nano-copper particles is attributed to their ability to generate oxidative stress on bacterial cells and create hydrogen peroxide that can kill the microbe. Furthermore, nano-copper particles interfere with proteins that are responsible for vital cellular functions or processes in the microbial cells.

In the preferred embodiment of the invention, the resonance antimicrobial coating composition comprises 0.01% to 30.0% by weight of the nano-sized metal oxide. More preferably, nano-sized silver oxide and/or copper oxide constitute 0.1% to 5.0% by weight of the composition. The amount of energy transferred to induce resonance may not be sufficient for less than 0.01% by weight of metal oxide. However, any amount more than 30.0% by weight of metal oxide would not provide any additional advantageous effect.

It is important that the metal oxide particles, particularly the silver and/or copper particles, in the composition are in the nano-sized range so as to provide bigger surface area for contact with microorganisms and capturing, holding, and releasing of vibration energy as compared to common micro-sized metal oxide particles. In an exemplary embodiment of the invention, 1 gram of 10 nm silver oxide particles provide a contact surface area of approximately 100 m 2. Preferably, the metal oxide particles in the composition are 5 nm to 100 nm in size.

Pursuant to the preferred embodiment of the invention, the resonance antimicrobial coating composition comprises at least one ultraviolet light-assisted photocatalyst selected from the group consisting of titanium oxide, titanium dioxide, tungsten oxide, tungsten trioxide, zinc oxide, and any combination thereof. The ultraviolet light-assisted photocatalyst is responsive to an ultraviolet or fluorescent light source to oxidize viruses, bacteria, mold, fungi, odor, volatile organic compounds and toxic gases. Preferably, the composition comprises 0.01% to 30.0% by weight of an ultraviolet light-assisted photocatalyst.

Photocatalysis is initiated when ultraviolet light-assisted photocatalyst in the composition is exposed to ultraviolet or fluorescent light rays. This is known as an optical solid surface or interface reaction. Upon activation of the photocatalyst, a repetitive oxidation-reduction (redox) reaction occurs at the surface of the ultraviolet light-assisted photocatalyst. Air containing oxygen and water vapor is required for the redox reaction.

During the photocatalysis process, the photocatalyst absorbs ultraviolet or fluorescent rays to generate electrons and positive-charged holes. The greater the generation of electrons (e⁻) and positive-charged holes (h⁺), the higher the reaction effect is. The reaction of photocatalysis is shown as below:

TiO₂(photocatalyst)+hv(ultraviolet rays)→e⁻+h⁺

The generated positive holes have strong oxidization ability and by reacting with water molecules present on the surface of the photocatalyst, hydroxyl radicals (⋅OH) can be generated. The hydroxyl radicals are generated through a reaction as shown below:

h⁺+H₂O→OH+H⁺

The generated hydroxyl radicals oxidize organic contaminant compounds. In the presence of oxygen, radicals of the intermediates of organic contaminant compounds induce a radical chain reaction and consume oxygen. As a results, the organic contaminants compounds are decomposed and eventually turn into carbon dioxide and water.

On the other hand, the generated electrons produce superoxide anions (O₂⁻) by causing a reductive reaction with oxygen on the surface of the photocatalyst, in which the reaction is shown as below:

e⁻+O₂→O₂⁻

The superoxide anions form oxides by adhering to the intermediates of the oxidation reaction or turn into hydrogen peroxide (H₂O₂) and then into water. Free oxygen radical (—O) is also generated in the air and directly affects the carbon-carbon bond of organic matter.

Since organic matter is usually more oxidizable than water, therefore the positive-charged holes are more likely to oxidize the organic compounds. The recombination rate of both carriers, which are the holes and electrons, shall decrease when the concentration of organic matter is higher.

The resonance antimicrobial coating composition disclosed herein may further comprise a liquid carrier which is a solvent-based or water-based. Particularly, the particles of metal oxide are contained within a liquid carrier so that the resonance antimicrobial coating composition is readily to be applied and coated on a surface. The liquid carrier also acts as a medium of transferring energy from an energy source to the metal oxide or from the metal oxide to the atoms of the antimicrobial coating. Any kind of liquid carrier which does not react with the metal oxide can be used. Preferably, the liquid carrier is a silicone oil or an alcohol or a water or a mixture thereof. More preferably, the alcohol can be selected from isopropanol, methanol, or ethanol, whilst the silicone oil can be selected from hexamethyl disiloxane, octamethyl trisiloxane, decamethylcyclo pentasiloxane, polydimethyl siloxane or octamethylcyclo tetrasiloxane. When a surface is coated with the composition, the presence of silicone oil also provides the surface with a smooth appearance as well as anti-stick characteristics so that dust or other solid impurities will not adhere to the surface. Preferably, the composition comprises 75% to 94% by weight of liquid carrier.

The resonance antimicrobial coating composition disclosed herein may further comprise a binder. A binder is needed to ensure the coating binds well to the surface to be coated. Preferably, the binder is a silane or monopolymer or copolymer. More preferably, the silane is an alkyl silane. The alkyl silane can be selected from methyl silane, dimethydiethoxysilane, tetraethoxysilane, linear dialkylsilane, fluorinated alkyl silane, or cyclic alkylsilane. Any silane binder which can render the composition be cured at room temperature and reduced curing time can be used. Preferably, the composition comprises 0.01% to 30% by weight of binder.

In addition to the carrier and binder, the resonance antimicrobial coating composition disclosed herein may further comprise a surface additive. Surface additive is added to further enhance binding of the coating to the surface to be coated. Preferably, the surface additive is an acid to decrease the pH of the composition. When the composition is coated on the surface, the acidic composition may slightly etch the surface and form bonds between the composition and the surface. It shall be noted that the amount of acid added shall not be high to the extent that the pH of the composition falls below 5 or become strongly acidic. It is preferred that the composition has a pH ranging from 5 to 6 which effectively enhance binding of the coating without causing any corrosion to any part of the device. Preferably, the acid can be selected from sulphuric acid, phosphoric acid, nitric acid, or hydrochloric acid.

An alkaline composition is not preferred as it may render the coating to be easily detached from the surface. Preferably, the composition comprises 0.1% to 8% by weight of surface additive. More preferably, the composition comprises less than 2% by weight of surface additive.

According to the preferred embodiment of the invention, atoms in the resonance antimicrobial coating composition hold sufficient energy which causes them to vibrate at a predetermined frequency for a period of time. Preferably, the atoms are in a state of energy excitation that vibrate at a frequency of 0.5 kHz to 500 kHz. Particularly, the atoms have been subjected to a resonance frequency bombardment with a vibration force at the frequency for at least 24 hours. When any surface is coated with the resonance antimicrobial coating composition, the atoms of the composition transfer the vibration energy in all direction at a distance ranging from 10 mm to 20 mm to the microorganisms on the surface. Accordingly, the cell membranes or cell walls of the microorganisms are induced to vibrate at a similar frequency. Particularly, the microbial cell membranes or cell walls are vibrating at their natural frequency where resonance occurs which eventually causing them to shatter and break apart.

Resting stages of microorganisms, particularly bacterial endospores, fungal spores and protozoan cysts, are generally more resistant to photocatalytic disinfection than the vegetative forms, possibly due to the increased cell wall thickness. Nevertheless, when atoms of the resonance antimicrobial coating composition are excited to vibrate at a frequency of 0.5 kHz to 500 kHz and the vibration energy is transferred to the microorganisms at resting stages, damage or shattering of the microbial cell walls becomes easier and more effective, hence the resistance of these microorganisms to photocatalytic disinfection can be reduced.

Besides, close contact between the microorganisms and the photocatalyst increases the extent of oxidative damage. Photocatalyst immobilized on surfaces, e.g. on thin layers or films, is less active than suspended photocatalyst. This is probably due to reduced contact between the photocatalyst particles and the microbial cells on the surface as well as a reduced surface area for reactive oxygen species (ROS) production. However, the oxidative damage caused by photocatalyst is improved when the photocatalyst is excited to vibrate at a frequency of 0.5 kHz to 500 kHz. In the preferred embodiment of the invention, upon application of the resonance antimicrobial coating composition over a surface, the ultraviolet light-assisted photocatalyst contained therein is immobilized. Yet, as the photocatalyst particles in the composition are excited to vibrate at a frequency of 0.5 kHz to 500 kHz, the oxidative damage caused by the immobilized photocatalyst is at least equivalent to that of suspended photocatalyst.

The resonance antimicrobial coating composition described in any of the preceding description can be produced with the following method. Components of the composition such as metal oxide, ultraviolet or fluorescent light-assisted photocatalyst, binder, liquid carrier, and surface additive are homogeneously mixed one at a time. The order of mixing is preferred to be binder, surface additive, liquid carrier, metal oxide and ultraviolet or fluorescent light-assisted photocatalyst. It shall be noted that metal oxide shall not be added before silane or mono-polymer or copolymer in order to achieve a homogeneous mixture. More preferably, the composition is homogenised by an ultrasonic mixer operating at a frequency of 20 kHz to 100 kHz for at least 1 hour. However, it is not necessary to mix the composition for more than 2 hours to achieve a homogeneous mixture. Any other method of homogenising the mixture can be adopted. During the homogenisation, nano particulates metal oxide can be further broken down into smaller size with a higher surface area to capture, hold, and release the vibration energy.

Subsequently, the mixture is subjected to bombardment with a vibration force at a frequency of 0.5 kHz to 500 KHz for at least 24 hours to store energy within the nano-particles. The vibration force can be provided in any form. Preferably, the vibration force is provided by an ultrasonic means. Sufficiently long period of bombardment time is required so as to allow atoms of the composition, particularly atoms of the metal oxide and ultraviolet or fluorescent light-assisted photocatalyst, to capture and hold the energy from the vibration force for a period of time. Atoms of the composition with the energy are excited to vibrate vigorously for a period of time at a frequency similar to the frequency of the vibration force.

The homogenisation step and bombardment step can be in a single operation in which only ultrasonic treatment is utilised. After mixing the composition, the mixture is subjected to ultrasonic treatment where homogenisation and energy capturing occur simultaneously. The ultrasonic frequency is preferably at 0.5 kHz to 500 KHz and the treatment is preferably last for at least 24 hours. However, composition produced using single operation method is prone to have phase separation. Although phase separation may not affect the performance of the composition, the aesthetic view of the composition may not be welcome by the user.

Alternatively, the homogenisation step and bombardment step can be in two separate operations even only ultrasonic treatment is utilised. The binder, surface additive, and liquid carrier are mixed and homogenise by ultrasonic mixer at a frequency of 20 kHz to 100 kHz for at least 1 hour, preferably not more than 2 hours. Subsequently, metal oxide is added to the homogenised mixture. The resultant mixture is subjected to ultrasonic treatment at a frequency of 0.5 kHz to 500 kHz for at least 24 hours.

Although the invention has been described and illustrated in detail, it is to be understood that the same is by the way of illustration and example, and is not to be taken by way of limitation. The scope of the invention are to be limited only by the terms of the appended claims.

### EXAMPLES

An example is provided below to illustrate different aspects and embodiments of the invention. The example is not intended in any way to limit the disclosed invention, which is limited only by the claims.

### Example 1

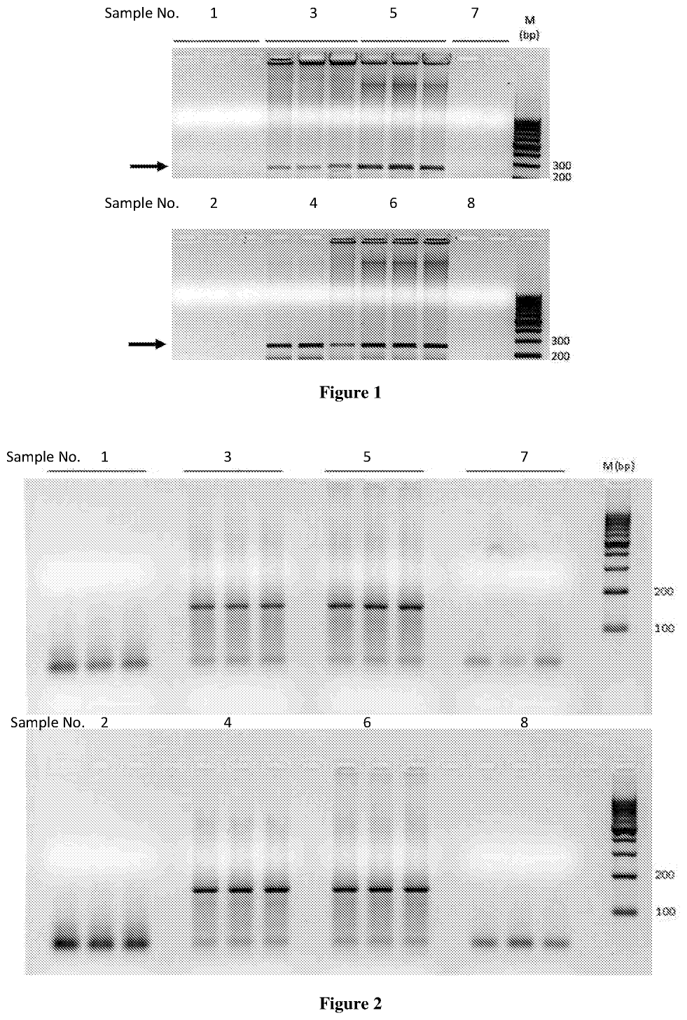

Evaluation of the antimicrobial effect of the disclosed resonance antimicrobial coating composition against Coronavirus 043 (CoV-O43) 8 test samples are prepared in triplicate according to Table 1. The test virus being used for the test is Coronavirus 043 (CoV-O43). Concentration of the test virus in each sample is 10⁴ pfu/ml. The volume ratio of the resonance antimicrobial coating composition to the test virus in samples no. 1 and 2 is 3:1. The test samples are kept at appropriate temperature for the duration of contact time specified in Table 1. Samples no. 1 to 4 are exposed to white/visible light whereas samples no. 5 to 8 are not subjected to any light exposure during the specified contact time. Immediately after the specified contact time, the test samples are subjected to viral nucleotide extraction and a pan-coronavirus reverse transcription polymerase chain reaction (RT-PCR) assay for detection of test virus in the test samples. The PCR products of each test sample are subjected to agarose gel electrophoresis. Presence of PCR product of 251 bp in the agarose gel indicates presence of test virus in the test sample. The test results are shown in Table 1 and FIG. 1.

TABLE 1

| | | | Duration of | |
|---|---|---|---|---|
| | | Visible | contact time | Detection of |
| Sample | | light | with test | PCR product |
| No. | Sample content | Exposure | virus (mins) | of 251 bp |
| 1 | Test virus, | Yes | 5 | No |
| 2 | Resonance | Yes | 60 | No |
| | antimicrobial | | | |
| | coating composition | | | |
| 3 | Test virus | Yes | 5 | Yes |
| 4 | | Yes | 60 | Yes |
| 5 | Test virus (Positve | No | 5 | Yes |
| 6 | control) | No | 60 | Yes |
| 7 | Empty (Negative | No | 5 | No |
| 8 | control) | No | 60 | No |

Refer to Table 1 and FIG. 1, no PCR product of 251 bp is detected in both samples no. 1 and 2, indicating the test virus (Coronavirus 043, CoV-O43) and its viral RNA in these samples are efficiently degraded by the resonance antimicrobial coating composition disclosed herein through contact killing. Furthermore, the test results show that the resonance antimicrobial coating composition is effective against CoV-O43 after a period as short as 5 mins. A quick surface disinfecting effect can be achieved by using the resonance antimicrobial coating composition disclosed herein.

### Example 2

Evaluation of the antimicrobial effect of the disclosed resonance antimicrobial coating composition against Enterovirus A71 (EV-A71)

8 test samples are prepared in triplicate according to Table 2. The test virus being used for the test is Enterovirus A71 (EV-A71). Concentration of the test virus in each sample is 10⁵ pfu/ml. The volume ratio of the resonance antimicrobial coating composition to the test virus in samples no. 1 and 2 is 3:1. The test samples are kept at appropriate temperature for the duration of contact time specified in Table 2. Samples no. 1 to 4 are exposed to white/visible light whereas samples no. 5 to 8 are not subjected to any light exposure during the specified contact time. Immediately after the specified contact time, the test samples are subjected to viral nucleotide extraction and a pan-enterovirus reverse transcription polymerase chain reaction (RT-PCR) assay for detection of test virus in the test samples. The PCR products of each test sample are subjected to agarose gel electrophoresis. Presence of PCR product of 154 bp in the agarose gel indicates presence of test virus in the test sample. The test results are shown in Table 2 and FIG. 2.

TABLE 2

| | | | Duration of | |
|---|---|---|---|---|
| | | Visible | contact time | Detection of |
| Sample | | light | with test | PCR product |
| No. | Sample content | Exposure | virus (mins) | of 154 bp |
| 1 | Test virus, | Yes | 5 | No |
| 2 | Resonance | Yes | 60 | No |
| | antimicrobial | | | |
| | coating composition | | | |
| 3 | Test virus | Yes | 5 | Yes |
| 4 | | Yes | 60 | Yes |
| 5 | Test virus (Positve | No | 5 | Yes |
| 6 | control) | No | 60 | Yes |
| 7 | Empty (Negative | No | 5 | No |
| 8 | control) | No | 60 | No |

Refer to Table 2 and FIG. 2, no PCR product of 154 bp is detected in both samples no. 1 and 2, indicating the test virus (Enterovirus A71, EV-A71) and its viral RNA in these samples are efficiently degraded by the resonance antimicrobial coating composition disclosed herein through contact killing. Furthermore, the test results show that the resonance antimicrobial coating composition is effective against EV-A71 after a period as short as 5 mins. A quick surface disinfecting effect can be achieved by using the resonance antimicrobial coating composition disclosed herein.

### Example 3

Evaluation of the antimicrobial effect of the disclosed resonance antimicrobial coating composition against Coxsackievirus A6 (CVA-6) and Coxsackievirus A16 (CVA-16)

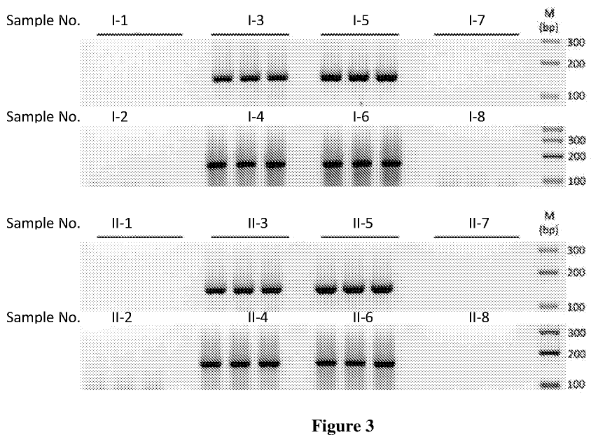

Two sets of 8 test samples are prepared in triplicate according to Table 3. The test virus being used in the first set (Set I) of test samples is Coxsackievirus A6 (CVA-6) while the one being used in the second set (Set II) of test samples is Coxsackievirus A16 (CVA-16). Concentration of the test virus in each sample is 10⁵ pfu/ml. The volume ratio of the resonance antimicrobial coating composition to the test virus in samples no. I-1, I-2, II-1 and II-2 is 3:1. The test samples are kept at appropriate temperature for the duration of contact time specified in Table 3. In each set I and II, samples no. 1 to 4 are exposed to white/visible light whereas samples no. 5 to 8 are not subjected to any light exposure during the specified contact time. Immediately after the specified contact time, the test samples are subjected to viral nucleotide extraction and a pan-enterovirus reverse transcription polymerase chain reaction (RT-PCR) assay for detection of test virus in the test samples. The PCR products of each test sample are subjected to agarose gel electrophoresis. Presence of PCR product of 154 bp in the agarose gel indicates presence of test virus in the test sample. The test results are shown in Table 3 and FIG. 3.

TABLE 3

| | | | | Duration of | |
|---|---|---|---|---|---|
| | Sam- | | Visible | contact time | Detection of |
| | ple | | light | with test | PCR product |
| Set | No. | Sample content | Exposure | virus (mins) | of 154 bp |
| I | 1 | Test virus (CVA-6), | Yes | 5 | No |
| | 2 | Resonance | Yes | 60 | No |
| | | antimicrobial | | | |
| | | coating | | | |
| | | composition | | | |
| | 3 | Test virus (CVA-6) | Yes | 5 | Yes |
| | 4 | | Yes | 60 | Yes |
| | 5 | Test virus (CVA-6) | No | 5 | Yes |
| | 6 | (Positve control) | No | 60 | Yes |
| | 7 | Empty (Negative | No | 5 | No |
| | 8 | control) | No | 60 | No |
| II | 1 | Test virus (CVA- | Yes | 5 | No |
| | 2 | 16), Resonance | Yes | 60 | No |
| | | antimicrobial | | | |
| | | coating | | | |
| | | composition | | | |
| | 3 | Test virus (CVA- | Yes | 5 | Yes |
| | 4 | 16) | Yes | 60 | Yes |
| | 5 | Test virus (CVA- | No | 5 | Yes |
| | 6 | 16) (Positve | No | 60 | Yes |
| | | control) | | | |
| | 7 | Empty (Negative | No | 5 | No |
| | 8 | control) | No | 60 | No |

Refer to Table 3 and FIG. 3, no PCR product of 154 bp is detected in the samples no. I-1, I-2, II-1 and II-2, indicating the test viruses (Coxsackievirus A6, CVA-6 and Coxsackievirus A16, CVA-16) and their viral RNA in these samples are efficiently degraded by the resonance antimicrobial coating composition disclosed herein through contact killing. Furthermore, the test results show that the resonance antimicrobial coating composition is effective against CVA-6 and CVA-16 after a period as short as 5 mins. A quick surface disinfecting effect can be achieved by using the resonance antimicrobial coating composition disclosed herein.

### Example 4

Efficacy test of the disclosed resonance antimicrobial coating composition against Escherichia coli and Staphylococcus aureus

A disinfectant efficacy test is performed using a resonance antimicrobial coating composition comprising nano-sized copper oxide particles as active ingredients (test composition) disclosed herein. The test is performed using in house method with reference to The United States Pharmacopeia 51 in which test microorganisms such as Escherichia coli ATCC 8739 and Staphylococcus aureus ATCC 6538 are introduced to the test composition. The inoculums of test microorganisms are prepared on petri dishes at the range about 10⁴ as shown in Table 4 and the test composition is coated onto inner side of petri dish lids to emit fusion resonance frequency on the test microorganisms with a distance of 1 cm apart. The petri dish are analysed after a specific time to determine the number of remaining viable microorganisms. The results are shown in Table 4 below.

TABLE 4

| | | Concentration | Concentration | |
|---|---|---|---|---|
| Parameters/ | Treat- | of challenged | of microbes | Microbial |
| challenged | ment | microbes | recovered | reduction |
| bacteria | time | (cfu/ml) | (cfu/ml) | (%) |
| Escherichia coli | 5 min | 4.3 × 10⁴ | 1.2 × 10³ | 97.21 |
| ATCC 8739 | 10 min | 4.3 × 10⁴ | 1.0 × 10³ | 97.67 |
| Staphylococcus | 5 min | 4.8 × 10⁴ | 1.2 × 10⁴ | 75.00 |
| aureus ATCC | 10 min | 4.8 × 10⁴ | 1.0 × 10³ | 97.92 |
| 6538 | | | | |

As shown in Table 4, the resonance antimicrobial coating composition disclosed herein is capable of effectively killing bacteria such as Escherichia coli and Staphylococcus aureus through non-contact killing. Particularly, at least 75% of the bacteria on a surface is eliminated 5 mins after the composition is applied over a nearby surface.

### Example 5

Efficacy test of the disclosed resonance antimicrobial coating composition against severe acute respiratory syndrome coronavirus 2 (SARS-CoV-2) responsible for coronavirus disease 2019 (COVID-19)

Cells and Viruses

The test virus, severe acute respiratory syndrome coronavirus 2 (SARS-CoV-2), was isolated, propagated and maintained in Vero E6 cells at Tropical Infectious Diseases Research and Education Center (TIDREC), University of Malaya, Malaysia. The Vero E6 cells were cultured in Dulbecco's Modified Eagle Medium (DMEM) (Gibco, Grand Island, New York, USA) supplemented with 10% fetal bovine serum (FBS). The cells were maintained at 37° C. with 5% carbon dioxide (CO₂). Viral titers were determined by microtitration using the Vero E6 cells and expressed in tissue culture infectious dose 50% (TCID₅₀/mL). When cytopathic effects (CPE) were evident under the microscope, the supernatant was harvested, clarified by centrifugation and stored at −80° C. until needed.

In Vitro Quantitative Suspension Assay

A resonance antimicrobial coating composition comprising titanium dioxide, silver ions and copper ions as active ingredients (test product A) and a resonance antimicrobial coating composition comprising copper ions as active ingredients (test product B) were tested against the test virus in accordance to the European Standard EN14476:2013/FprA1:2015 protocol. The experimental conditions are shown in Table 5. Test product A and test product B were tested undiluted and at a 2-fold dilution under two different conditions, namely dirty condition (3.0 g/l bovine serum albumin (BSA)+3 ml/l erythrocytes interfering substance) and clean condition (0.3 g/l BSA interfering substance), at 1 min, 5 min and 10 min contact time. The test mixtures comprised of 100 μl of interfering substance, 100 μl of virus suspension at concentration of 5.42×10⁵ TCID₅₀/mL, and 100 μl of test product A or 800 μl of test product B. After the specified contact time (1 min, 5 min and 10 min), virucidal activities of the test products were immediately suppressed by adding DMEM+2% FBS to the test mixtures and then the mixtures were diluted in 10 fold in ice-cold media (DMEM+2% FBS). The diluted test mixtures were added to the Vero E6 cells to determine TCID₅₀/mL. A virus control mixture was also assessed using distilled water in place of the test products for both dirty and clean conditions. The cells were incubated for 72 hours till the CPE developed. A mixture of paraformaldehyde and crystal violet was used to fix and stain the infected cells. The viral titers were determined using the Spearman-Karber method and expressed as tissue culture infectious dose 50% (TCID₅₀/mL). The virucidal activities of test product A and test product B were determined by the difference of the logarithmic titer of the virus control minus the logarithmic titer of the test virus (Δ log₁₀ TCID₅₀/mL). A reduction in viral titer of 4 log₁₀ (corresponding to an inactivation of ≥99.99%) was necessary for claiming virucidal activity of the test products.

TABLE 5

| | |
|---|---|
| Test temperature | 21.0° C. ± 1° C. |
| Test product's | Test product A: titanium dioxide, silver ion and |
| active ingredient(s) | copper ion |
| | Test product B: copper ion |
| Test product | Test product A: 5% |
| concentration | Test product B: 2% |
| Contact times | Contact time: 1 min, 5 min and 10 min |
| | Coated: 2 hour (Post-application) |
| Conditions | Clean condition: 0.3 g/l BSA |
| | Dirty condition: 3.0 g/l BSA + 3.0 ml/l human |
| | erythrocytes |
| Diluent for test | Distilled water |
| product A | |
| Temperature of | 37° C. ± 1° C., CO₂ incubator (5% CO₂) |
| incubation | |
| Test virus | SARS-CoV-2 |
| Test virus: source | Tropical Infectious Disease Research & Education |
| | Centre (TIDREC), University of Malaya, Malaysia |
| Test virus: number | 2 |
| of passages | |
| Cell line | Vero E6 |
| Cell line: source | ATCC |
| Cell line: number | Passages 22 |
| of passages | |

Suppression Assay

Suppression of virucidal acitivities of test product A and test product B was performed to accurately determine the virucidal activity of the test products at the specified contact time. Virucidal activity was suppressed by adding ice-cold DMEM+2% FBS to the test mixtures, and serially diluted 10-fold with cell culture medium. The suppression of virucidal acitivity of test products was assayed at 1 min exposure. As shown in Table 6, results from the suppression assay showed no difference in viral titers in the test mixtures compared to controls. This suggested that addition of cold media and serial dilution effectively suppressed the virucidal activity of the test products, resulting in no reduction in the viral titers.

TABLE 6

| | | | | Viral | |
|---|---|---|---|---|---|
| | | | Viral | titer | Difference |
| | | | titer | [after | in viral |
| *Contact | | | [control] | suppression] | titer |
| time | Interfering | Test | (TCID₅₀/ | (TCID₅₀/ | (TCID₅₀/ |
| (sec) | substance(s) | product | ml) | ml) | ml) |
| 60 | 0.3 g/l | A | 5.4 × 10⁵ | No inhibition | 0.00 |
| | BSA | B | 5.4 × 10⁵ | No inhibition | 0.00 |
| 60 | 3.0 g/l | A | 5.4 × 10⁵ | No inhibition | 0.00 |
| | BSA + | B | 5.4 × 10⁵ | No inhibition | 0.00 |
| | 3.0 ml/ | | | | |
| | l human | | | | |
| | erythrocytes | | | | |

*Undiluted mixture

Virucidal Activity of Test Products

The test products A and B were tested against the test virus in accordance to the European Standard EN14476:2013/FprA1:2015 protocol. Manifestation of virus cytopathic effects in cell cultures was determined by comparing the test product-treated samples against that of the control-treated samples. The viral titer in the control-treated samples under clean and dirty conditions are 5.42×10 5 TCID₅₀/ml. Both the test product A and test product B, when tested neat, achieved >5 log₁₀ reduction in viral titers when exposed for 1 min, 5 min and 10 min under both clean and dirty conditions as shown in Table 7.

TABLE 7

| | | Log₁₀ reduction in viral titers as compared to | | | | | |
|---|---|---|---|---|---|---|---|
| | | controls | | | | | |
| Test | Test | Clean condition | | | Dirty condition | | |
| virus | product | 1 min | 3 min | 10 min | 1 min | 3 min | 10 min |
| SARS- | A (undiluted) | >5.00 | >5.00 | >5.00 | >5.00 | >5.00 | >5.00 |
| CoV-2 | B (undiluted) | >5.00 | >5.00 | >5.00 | >5.00 | >5.00 | >5.00 |

The resonance antimicrobial coating composition comprising titanium dioxide, silver ion and copper ion as active ingredients (test product A) and the resonance antimicrobial coating composition comprising copper ion as active ingredients (test product B), when tested undiluted, demonstrated potent and rapid virucidal activity of ≥5 log₁₀ reduction in SARS-CoV-2 (test virus) viral titer in 1 min under both clean and dirty conditions. These findings suggest that the disclosed resonance antimicrobial coating compositions can kill 99.99% SARS-CoV-2 in 1 min through contact killing.

### Example 6

Efficacy test of the disclosed resonance antimicrobial coating composition against Escherichia coli, Staphylococcus aureus, Pseudomonas aeruginosa, Candida albican and Aspergillus brasiliensis

A disinfectant efficacy test is performed using a resonance antimicrobial coating composition comprising nano-sized copper oxide particles as active ingredients (test composition) disclosed herein. The test is performed using in house method with reference to The United States Pharmacopeia 51 as described in Example 4 in which test microorganisms such as Escherichia coli ATCC 8739, Staphylococcus aureus ATCC 6538, Pseudomonas aeruginosa ATCC 9027, Candida albican ATCC 10231, and Aspergillus brasiliensis ATCC 16404 are introduced to the test composition. The test parameters and results are shown in Table 8 below.

TABLE 8

| | | | Concentration | Concentration | |
|---|---|---|---|---|---|
| Parameters/ | Treat- | | of challenged | of microbes | Microbial |
| challenged | ment | | microbes | recovered | reduction |
| bacteria | time | | (cfu/ml) | (cfu/ml) | (%) |
| Escherichia | 1 | min | 3.0 × 10⁸ | ND < 10 | 99.99 |
| coli ATCC | 5 | min | 3.0 × 10⁸ | ND < 10 | 99.99 |
| 8739 | 10 | min | 3.0 × 10⁸ | ND < 10 | 99.99 |
| Staphylococcus | 1 | min | 4.6 × 10⁸ | ND < 10 | 99.99 |
| aureus ATCC | 5 | min | 4.6 × 10⁸ | ND < 10 | 99.99 |
| 6538 | 10 | min | 4.6 × 10⁸ | ND < 10 | 99.99 |
| Pseudomonas | 1 | min | 4.2 × 10⁸ | ND < 10 | 99.99 |
| aeruginosa | 5 | min | 4.2 × 10⁸ | ND < 10 | 99.99 |
| ATCC 9027 | 10 | min | 4.2 × 10⁸ | ND < 10 | 99.99 |
| Candida | 1 | min | 1.4 × 10⁶ | ND < 10 | 99.99 |
| albican | 5 | min | 1.4 × 10⁶ | ND < 10 | 99.99 |
| ATCC 10231 | 10 | min | 1.4 × 10⁶ | ND < 10 | 99.99 |
| Aspergillus | 1 | min | 2.0 × 10⁶ | ND < 10 | 99.99 |
| brasiliensis | 5 | min | 2.0 × 10⁶ | ND < 10 | 99.99 |
| ATCC 16404 | 10 | min | 2.0 × 10⁶ | ND < 10 | 99.99 |

The results shown in Table 8 shows that the resonance antimicrobial coating composition disclosed herein is capable of effectively killing bacteria such as Escherichia coli, Staphylococcus aureus, Pseudomonas aeruginosa, Candida albican and Aspergillus brasiliensis through non-contact killing. Particularly, 99.99% of the bacteria on a surface is eliminated 1 min after the composition is applied over a nearby surface.

### Example 7

Efficacy test of the disclosed resonance antimicrobial coating composition against Salmonella typhimurium, Staphylococcus aureus and Candida albican

A disinfectant efficacy test is performed using a resonance antimicrobial coating composition comprising titanium dioxide, silver oxide and copper sulfate as active ingredients (Test composition 1) and a disinfectant efficacy test is performed using a resonance antimicrobial coating composition comprising tourmaline as active ingredient (Test composition 2) disclosed herein. The test is performed using in house method with reference to The United States Pharmacopeia (USP) 41 in which test microorganisms such as Salmonella typhimurium ATCC 14028, Staphylococcus aureus ATCC 25923 and Candida albican ATCC 10231 are introduced to the undiluted test compositions. The test parameters and results for test composition 1 and test composition 2 are shown in Table 9 and Table 10, respectively.

TABLE 9

| | | Initial | | Count of | | Percent kill |
|---|---|---|---|---|---|---|
| | Contact | bacterial | | surviving test | | of test |
| Test | time | load | | microorganism, | Log | microorganisms |
| Microorganism | (min) | (cfu/ml) | log₁₀ | cfu/ml (log₁₀) | reduction | (%) |
| Salmonella | 5 | 6.1 × 10⁸ | 8.79 | <10 (1.0) | >7.79 | >99.9999 |
| typhimurium | 60 | | | <10 (1.0) | >7.79 | >99.9999 |
| ATCC 14028 | | | | | | |
| Staphylococcus | 5 | 1.3 × 10⁹ | 9.11 | <10 (1.0) | >8.11 | >99.9999 |
| aureus ATCC | 60 | | | <10 (1.0) | >8.11 | >99.9999 |
| 25923 | | | | | | |
| Candida | 5 | 4.1 × 10⁶ | 6.61 | <10 (1.0) | >5.61 | >99.9999 |
| albican ATCC | 60 | | | <10 (1.0) | >5.61 | >99.9999 |
| 10231 | | | | | | |

TABLE 10

| | | Initial | | Count of | | Percent kill |
|---|---|---|---|---|---|---|
| | Contact | bacterial | | surviving test | | of test |
| Test | time | load | | microorganism, | Log | microorganisms |
| Microorganism | (min) | (cfu/ml) | log₁₀ | cfu/ml (log₁₀) | reduction | (%) |
| Salmonella | 5 | 6.1 × 10⁸ | 8.79 | <10 (1.0) | >7.79 | >99.9999 |
| typhimurium | 60 | | | <10 (1.0) | >7.79 | >99.9999 |
| ATCC 14028 | | | | | | |
| Staphylococcus | 5 | 1.3 × 10⁹ | 9.11 | <10 (1.0) | >8.11 | >99.9999 |
| aureus ATCC | 60 | | | <10 (1.0) | >8.11 | >99.9999 |
| 25923 | | | | | | |
| Candida | 5 | 4.1 × 10⁶ | 6.61 | <10 (1.0) | >5.61 | >99.9999 |
| albican ATCC | 60 | | | <10 (1.0) | >5.61 | >99.9999 |
| 10231 | | | | | | |

The results shown in Tables 9 and 10 show that the resonance antimicrobial coating composition disclosed herein is capable of effectively killing bacteria such as Salmonella typhimurium, Staphylococcus aureus, and Candida albican killing after 5 minutes.

1.-8. (canceled)

9. An antimicrobial coating composition for use in surface disinfection, comprising:
a nano-sized metal oxide selected from the group consisting of silver oxide, copper oxide, and combinations thereof; and
an ultraviolet or fluorescent light-assisted photocatalyst selected from the group consisting of titanium oxide, titanium dioxide, tungsten oxide, tungsten trioxide, zinc oxide, and combinations thereof;
wherein atoms of the composition are subjected to bombardment with a vibration force at a frequency of 0.5 kHz to 500 kHz for at least 24 hours.

10. The antimicrobial coating composition as claimed in claim 9, further comprising any one or any combination of a nano-sized silica and a nano-sized metal oxide, wherein the nano-sized metal oxide is selected from the group consisting of titanium oxide, titanium dioxide, tungsten oxide, tungsten trioxide, zinc oxide, calcium oxide, magnesium oxide, tin oxide, gold oxide, and combinations thereof.

11. The antimicrobial coating composition as claimed in claim 10, wherein the composition comprises from 0.01% to 30.0% by weight of the nano-sized metal oxide.

12. The antimicrobial coating composition as claimed in claim 11, wherein the nano-sized metal oxide has a particle size ranging from 5 nm to 50 nm.

13. The antimicrobial coating composition as claimed in claim 12, wherein the composition comprises 0.01% to 30.0% by weight of the ultraviolet or fluorescent light-assisted photocatalyst.

14. The antimicrobial coating composition as claimed in claim 13, further comprising any one of a binder, a liquid carrier, and a surface additive.

15. A method of preparing an antimicrobial coating composition for use in surface disinfection, comprising the steps of:
providing a nano-sized metal oxide selected from the group consisting of silver oxide, copper oxide, and combinations thereof; and
providing an ultraviolet or fluorescent light-assisted photocatalyst selected from the group consisting of titanium oxide, titanium dioxide, tungsten oxide, tungsten trioxide, zinc oxide, and combinations thereof;
wherein atoms of the composition are subjected to bombardment with a vibration force at a frequency of 0.5 kHz to 500 kHz for at least 24 hours.
